(19) United States
(12) Patent Application Publication
(10) Pub. No.: US 2006/0078616 A1
(43) Pub. Date: Apr. 13, 2006

(54) Thermoreversible pharmaceutical formulation for anti-microbial agents comprising poloxamer polymers and hydroxy fatty acid ester of polyethylene glycol

(76) Inventors: Dawaye A. Georgewill, Brampton (CA); Maxine G. Moldenhauer, Acton (CA); Terry Feldman, Richmond Hill (CA)

Correspondence Address: TARO PHARMACEUTICALS, 5 SKYLINE DRIVE, HAWTHORNE, NY, 10532, US

(21) Appl. No.: 11/204,522

(22) Filed: Aug. 16, 2005

Related U.S. Application Data
(60) Provisional application No. 60/605,429, filed on Aug. 30, 2004.

(51) Int. Cl. A61K 31/7048; A61K 9/14

(52) U.S. Cl. 424/486; 424/489; 514/28

(57) ABSTRACT

The present invention provides a pharmaceutical formulation having thermoreversible properties, comprising: a) an anti-microbial agent; b) a poloxamer mixture containing at least two poloxamer polymers; and c) a hydroxy fatty acid ester of polyethylene glycol, wherein the formulation is a solid at room temperature and is a liquid-gel at body temperature. The thermoreversible pharmaceutical formulation has a viscosity of about 8,500 cP to about 400,000 cP at room temperature, and a viscosity of about 1,000 cP to about 8,000 cP at body temperature and exhibits a hysteresis loop behavior. The present invention further provides a process of preparing as well as a method of treating a microbial infection in a mammal using the same.

### CROSS REFERENCE TO RELATED APPLICATION

This application claims the benefit under 35 U.S.C. § 1.119(e) of Provisional Application Ser. No. 60/605,429, filed Aug. 30, 2004, the disclosure of which is incorporated by reference in its entirety herein.

### FIELD OF THE INVENTION

The present invention relates to a pharmaceutical formulation with thermoreversible properties suitable for vaginal and rectal delivery of drugs. In particular, the present pharmaceutical formulation relates to a mixture of at least two poloxamer polymers, a polyethylene-glycol fatty acid ester and an anti-microbial agent such as clindamycin phosphate.

### BACKGROUND OF THE INVENTION

Numerous pharmaceutical formulation systems exist for vaginal or rectal delivery of drugs. Early in the history of suppository preparation, Cocoa Butter (Theobroma Oil) was the only available fatty material used. Suppository formulations using lipophilic tri-glyceride fatty acid vehicles (such as Hard fat) are currently available and represent a main drug delivery choice. For example, U.S. Pat. No. 6,495,157 describes a vaginal suppository for clindamycin phosphate sold under the trademark Cleocin®, currently marketed by Pharmacia and Upjohn as the only clindamycin phosphate suppository product. The formulation, fabricated with Hard fat which is a common tri-glyceride material, suffers from defects common to such suppository products including instability of active pharmaceutical ingredient, poor absorption, limited effectiveness due to frequent discharge, and messy leakage from the vaginal vault. These defects relate to the fact that Hard fat is present as a solid form at room temperature but rapidly (within 20 min) melts into an oil when inserted into a human body. The recommended indication of Cleocin® includes advising patients to lie down immediately after application for several hours to prevent leakage. U.S. Pat. No. 6,416,779 cites additional advantage of this lipophilic tri-glyceride fatty acid base formulation to include “loss of drug due to such leaking, uncertainty of the amount of the drug delivered and general feeling of non-sanitary conditions which occur during such treatment.”

Other formulation vehicles have been introduced for suppository dosage forms. In this regard, hydrophilic polyethylene glycol-based vehicles consisting of mixtures of various PEG grades with molecular weight in the range of 1,000 to 6,000 have been reported. However, the hydrophilic polyethylene glycol-base vehicles also suffer leakage and instability problems as well as elicit tissue irritation.

Specific ingredients present within the pharmaceutical formulation may affect the physico-chemical properties of the pharmaceutical formulation. There has been research on the thermoreversible activity of poloxamers (i.e., poly(oxyethylene)-poly(oxypropylene)-poly(oxyethylene)) tri-block copolymer and the fabricating of gel systems that may be of use in pharmaceutical applications. Water-based aqueous poloxamer solutions exist as a liquid at temperatures below 20° C. and transforms into solid state (i.e., gel) at higher temperatures (i.e., 40° C.). Yoon, Sung June et al. (U.S. Pat. No. 6,488,954), Choi, H-G et al. (Int. J. Pharmaceutics 165: 33-44, 1998), Kim, C-K et al. (Int. J. Pharmaceutics 174: 201-207, 1998): Ryu, J-M et al. (J. Controlled Release 59: 163-172, 1999), and Chang, J Y et al. (Int. J. Pharmaceutics 241: 155-163, 2002) describe thermoreversible property of poloxamer and its potential use as liquid suppositories for anorectal application. In these liquid suppositories, the poloxamer-based formulation is present as a liquid at room temperature and becomes solid when insert into a human body. To ensure complete dissolution of the poloxamers during the preparation, cold temperature conditions, such as 4-10° C., are employed (See, Eur. J. Pharma. Sci. 17: 161-167, 2002). Accordingly, the formulation is extremely cumbersome for manufacturing and storage. These preparations also suffer disadvantages common to liquid formulations including sedimentation of suspended particles and caking during storage, which may adversely affect accurate dosing of the required amount of drug substance during application.

U.S. Pat. No. 4,478,822 describes an aqueous thermosetting vehicle useful for the delivery of pharmacologically active medicament to a body cavity consisting of a clear liquid with physiological properties and forms a semi-solid gel at human body temperature. The delivery system is vulnerable to deficiencies generally inherent with liquid dosage forms during manufacture and storage, and chemical instability.

D'Cruz et al. (Biol. Reproduction 69: 1843-1851, 2003) discloses using a composition comprising of an anti-HIV agent (Stampidine), polyethylene glycol 400, polyethylene glycol fatty acid esters and Tween-80 (polyoxyethylene sorbitan monooleate). With two components of the composition being liquids and one component of the composition being pasty, the composition resembles an emulsion. The composition is devoid of mucoadhesive properties since none of the components are known to have such properties.

There is a continuing need for a thermoreversible pharmaceutical formulation comprising an anti-microbial agent suitable for vaginal or rectal administration without major leakage problems and good stability. It is desirable to prepare a pharmaceutical formulation wherein the formulation is a solid at room temperature and turns into a liquid-gel at body temperature.

### SUMMARY OF THE INVENTION

The present invention provides a thermoreversible pharmaceutical composition comprising an anti-microbial agent, a mixture of a first poloxamer polymer and a second poloxamer polymer, and a hydroxy fatty acid ester of polyethylene glycol as a vehicle based formulation where the formulation is a solid at room temperature (e.g., about 25° C.) and turns into a liquid gel when exposed to body temperature (e.g., about 37° C.). The present thermoreversible pharmaceutical formulation is suitable for the delivery of anti-microbial agents such as clindamycin phosphate to the body cavity.

Accordingly, the present invention provides a pharmaceutical formulation having a thermoreversible property, comprising:
a) an anti-microbial agent;
b) a poloxamer mixture containing a first poloxamer polymer and a second poloxamer polymer, wherein the first poloxamer polymer and the second poloxamer polymer are not the same, and wherein the first and the second poloxamer polymers are polymers represented by the chemical structure of:
   HO—(C₂H₄O)ₐ(C₃H₆O)_{b}(C₂H₄O)ₐ—H,
   where 101≦a≦80 and 56≦b≦27, and “a” and “b” denote the number of poly-oxyethylene and poly-oxypropylene units, respectively; and
c) a hydroxy fatty acid ester of polyethylene glycol,
wherein the pharmaceutical formulation having a viscosity of about 8,500 cP to about 400,000 cP at room temperature, a viscosity of about 1,000 cP to about 8,000 cP at body temperature, and exhibiting a hysteresis loop behavior.

Preferably, the pharmaceutical formulation has a viscosity of about 8,500 cP to about 25,000 cP at room temperature, and a viscosity of about 1,000 cP to about 5,000 cP at body temperature.

More preferably, the pharmaceutical formulation has a viscosity of about 8,500 cP to about 25,000 cP at room temperature and a viscosity of about 1,500 cP to about 3,000 cP at body temperature.

Preferably, the anti-microbial agent is selected from the group consisting of an anti-bacterial agent and an anti-fungal agent. Preferably, the anti-bacterial agent is at least one compound selected from the group consisting of clindamycin, metronidazole, mupirocin, bacitracin, neomycin sulphate. More preferably, the anti-bacterial agent is clindamycin. Most preferably, the anti-bacterial agent is clindamycin phosphate.

Preferably, the anti-fungal agent is at least one compound selected from the group consisting of clotrimzole, fluconazole, flucytosine, itraconazole, ketoconazole, miconazole, ciclopirox, econazole, nystatin, oxiconazole, terbinafine HCl, tioconazole, butoconazle, terconazole, miconazole nitrate, metronidazole, isoconazole nitrate, and tolnaftate. More preferably, the anti-fungal agent is clotrimazole.

Preferably, the anti-microbial agent is present in the amount of about 0.1 wt % to about 10 wt %. More preferably, the anti-microbial agent is present in the amount of about 2 wt % to about 5 wt %. More preferably, the anti-microbial agent is present in the amount of about 4.8 wt Preferably, the first poloxamer polymer is a poloxamer polymer selected from the group consisting of a copolymer of ethylene oxide and a copolymer of propylene oxide, wherein the poloxamer polymer is represented by the chemical structure of HO—(C₂H₄O)ₐ(C₃H₆O)_{b}(C₂H₄O)ₐ—H, where 101≦a≦80 and 56≦b≦27, and “a” and “b” denote the number of poly-oxyethylene and poly-oxypropylene units, respectively.

Preferably, the second poloxamer polymer is a poloxamer polymer selected from the group consisting of a copolymer of ethylene oxide and a copolymer of propylene oxide, wherein the poloxamer polymer is represented by the chemical structure of HO—(C₂H₄O)ₐ(C₃H₆O)_{b}(C₂H₄O)ₐ—H, where 101≦a≦80 and 56≦b≦27, and “a” and “b” denote the number of poly-oxyethylene and poly-oxypropylene units, respectively.

Preferably, the first poloxamer polymer is poloxamer 188, poloxamer 237, poloxamer 338, or poloxamer 407. More preferably, the first poloxamer is poloxamer 407 or poloxamer 188.

Preferably, the second poloxamer polymer is poloxamer 188, poloxamer 237, poloxamer 338, or poloxamer 407. More preferably, the second poloxamer is poloxamer 407 or poloxamer 188.

Preferably, the poloxamer polymers are present in the amount of about 5 wt % to about 30 wt %. More preferably, the poloxamer polymers are present in the amount of about 8 wt % to about 17 wt %. More preferably, the poloxamer polymers are present in the amount of about 16 wt %.

Preferably, the poloxamer mixture contains at least two poloxamer polymers. Preferably, the poloxamer mixture contains a first poloxamer polymer and a second poloxamer polymer, where the first poloxamer polymer is not the same as the second poloxamer polymer. Preferably, the first poloxamer polymer and the second poloxamer polymer is present in a wt/wt ratio of about 1:0.125 to about 1:1. More preferably, the first poloxamer polymer and the second poloxamer polymer is present in a wt/wt ratio of about 1:1.

Preferably, the hydroxy fatty acid ester of polyethylene glycol is polyethylene glycol 660 hydroxystearate. Preferably, the hydroxy fatty acid ester of polyethylene glycol is present in the amount of about 40 wt % to about 80 wt %. More preferably, the hydroxy fatty acid ester of polyethylene glycol is present in the amount of about 60 wt % to about 80 wt %. More preferably, the hydroxy fatty acid ester of polyethylene glycol is present in the amount of about 65 wt %.

Optionally, the pharmaceutical formulation further comprises water. Water may be present in the amount of about 5 wt % to about 30 wt %. Preferably, water is present in the amount of about 8 wt % to about 17 wt %.

The present invention further provides a process of preparing a pharmaceutical formulation having a thermoreversible property, comprising the steps of:
a) preparing a poloxamer mixture containing a first poloxamer and a second poloxamer;
b) preparing a molten solution of hydroxy fatty acid ester of polyethylene glycol;
c) adding the poloxamer mixture to the molten solution of hydroxy fatty acid ester of polyethylene glycol; and
d) adding an anti-microbial agent to form a thermoreversible pharmaceutical formulation.

Preferably, the poloxamer mixture is prepared by heating at a melting temperature of 65° C. More preferably, the heating is carried out at about 60° C. to about 70° C.

Preferably, the heating of hydroxy fatty acid ester of polyethylene glycol is carried out at about 60° C. to about 70° C.

The present invention further provides a method of treating a microbial infection in a mammal, comprising the step of administering to a mammal a thermoreversible pharmaceutical formulation containing a therapeutically effective amount of an anti-microbial agent. Preferably, the anti-microbial is clindamycin or clindamycin phosphate. Preferably, the mammal is a human. More preferably, the human is a female. Preferably, the pharmaceutical formulation is administered via intra-vaginally or intra-rectally. Preferably, pharmaceutical formulation is administered 2.5 grams/day.

The present invention further provides a pharmaceutical formulation having a thermoreversible property, comprising:
a) clindamycin phosphate;
b) a poloxamer mixture containing a first poloxamer polymer and a second poloxamer polymer, wherein the first poloxamer polymer and the second poloxamer are not the same, and wherein the first poloxamer polymer is poloxamer 407 and the second poloxamer polymer is poloxamer 188; and
c) polyethylene glycol 660 hydroxystearate,
   wherein the pharmaceutical formulation having a viscosity of about 8,500 cP to about 400,000 cP at room temperature, a viscosity of about 1,000 cP to about 8,000 cP at body temperature, and exhibiting a hysteresis loop behavior.

### BRIEF DESCRIPTION OF THE DIAGRAM

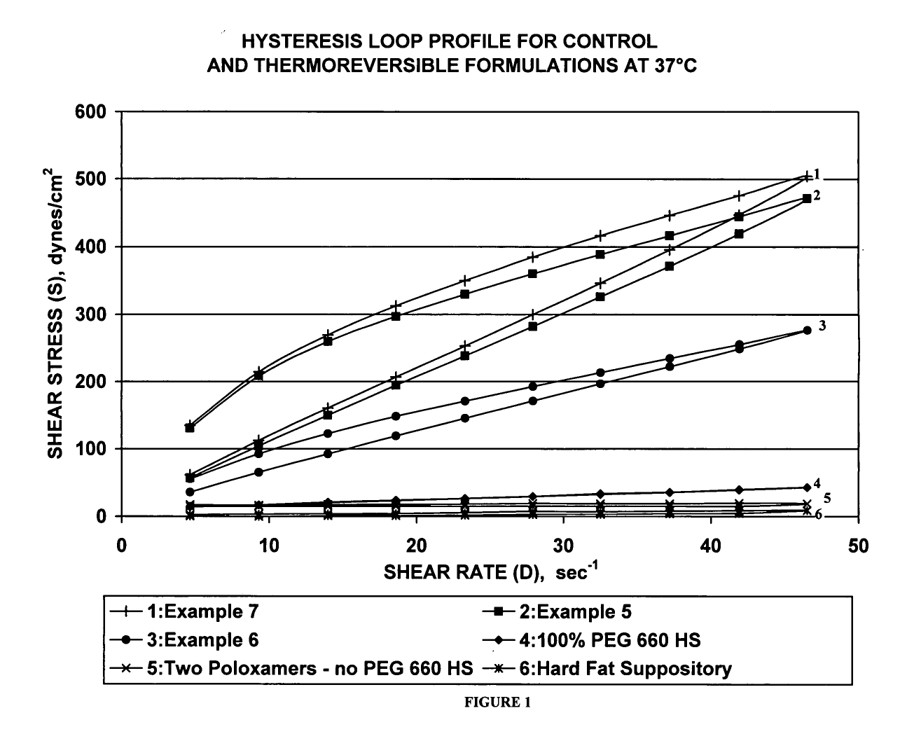

FIG. 1 depicts a hysteresis loop profile for control and thermoreversible pharmaceutical formulations at 37° C.

### DETAILED DESCRIPTION OF THE INVENTION

The following terms are defined: as used herein, the term “thermoreversible” refers to a physical property of a composition in which the composition can undergo a physical change of a solid state to a liquid-gel state repeatedly when exposed at different temperatures. Specifically, the present pharmaceutical formulation remains as a solid at room temperature and converts to a liquid-gel at body temperature, and the formulation can become solid again when the temperature returns to room temperature, and so on; the term “anti-microbial agent” refers to any chemical or biological agent that harms the growth of microorganisms; the term “hydroxy fatty acid ester of polyethylene glycol” refers mixture containing polyethylene glycol 660 esterified with a hydroxy fatty acid and un-esterified polyethylene glycol 660; the term “therapeutically effective amount” refers to a quantity of a pharmacologically active agent present in the composition and known to yield desired therapeutic outcome(s) when used as prescribed; the term “room temperature” refers to an ambient temperature of about 25° C., and it can range from 20° C. to 27° C.; the term “body temperature” refers to a temperature of core body of a human body of 37° C., and it can range from 35° C. to 39° C.; the term “liquid-gel” refers to a gel that is pourable within 10 minutes when slanted through 90 degrees; the term “solid” refers to a solid that is not pourable within 10 minutes when slanted through 90 degrees. For the purposes of the present invention, the solid has a viscosity range of about 8,500 cP to about 400,000 cP and the liquid-gel has a viscosity range of about 1,000 cP to about 8,000 cP. “Mammal” refers to a class of higher vertebrates comprising man and all other animals that nourish their young with milk secreted by mammary glands and have the skin usually more or less covered with hair; and “treating” is intended to encompass relieving, alleviating or eliminating at least one symptom of an infection condition in a mammal.

Unless otherwise indicated, as expressed in the present specification as well as in the set of claims as wt/wt, % (percentage) refers to % wt/wt.

It has been now been surprisingly discovered that a an improved pharmaceutical formulation with a thermoreversible property comprising an anti-microbial agent, a poloxamer mixture containing at least a first poloxamer polymer and a second poloxamer polymer, a hydroxy fatty acid ester of polyethylene-glycol, that is effective and safe in eliminating bacterial or yeast infections in a mammal. It has been discovered that the present pharmaceutical composition has a thermoreversible property that provides good local delivery of pharmacologic agents without leakage.

The present invention provides an improved pharmaceutical formulation having a thermoreversible property, and has the features of: a) being a solid with a viscosity of about 8,500 cP to about 400,000 cP at about 25° C.; b) being a liquid-gel with a viscosity of about 1,000 cP to about 8,000 cP at about 37° C.; and c) exhibiting a hysteresis loop behavior.

In accordance with the present invention, the thermoreversible pharmaceutical composition comprises an anti-microbial agent. The anti-microbial agent includes an anti-bacterial agent, an anti-fungal agent or anti-yeast agent.

Anti-bacterial agents include, but not limited to one compound selected from the group consisting of clindamycin, metronidazole, mupirocin, bacitracin, and neomycin sulphate. More preferably, the anti-bacterial agent is clindamycin. Most preferably, the anti-bacterial agent is clindamycin phosphate.

Anti-fungal agents include, but not limited to, clotrimzole, fluconazole, flucytosine, itraconazole, ketoconazole, miconazole, ciclopirox, econazole, nystatin, oxiconazole, terbinafine HCl, tioconazole, butoconazle, terconazole, miconazole nitrate, metronidazole, isoconazole nitrate, and tolnaftate. More preferably, the anti-fungal agent is clotrimazole.

Preferably, the anti-microbial agent is an anti-bacterial agent. Preferably, the anti-bacterial agent is clindamycin phosphate.

Clindamycin is also known as methyl 7-chloro-6,7,8-trideoxy-6-(1-methyl-trans-4-propyl-L-2-pyrrolidinecarboxamido)-1-thio-L-threo-.alpha.-D-galacto-octo-pyranoside or methyl 7-chloro-6,7,8-trideoxy-6-[[(1-methyl-4-propyl-2-pyrrolidinyl)carbonyl]amino]-1-thio-L-threo-.alpha.-D-galacto-octo-pyranoside and it is an effective anti-microbial agent. As used herein, the term “clindamycin” includes free-base clindamycin as well as the pharmaceutically acceptable salts and esters thereof.

The synthesis of clindamycin is well-known; for example, U.S. Pat. Nos. 3,969,516 and 3,475,407 describe the clindamycin preparation, the disclosure of which are incorporated herein by reference.

Examples of clindamycin pharmaceutically acceptable salts and esters include, but not limited to, clindamycin hydrochloride, clindamycin phosphate, and clindamycin palmitate. Preferably, clindamycin is clindamycin phosphate.

Preferably, the anti-microbial drug is present in an amount of about 0.1% weight to about 10% weight. Preferably, the amount is about 2% weight to about 5% weight. More preferably, the amount is about 4.8% weight.

The amount of the anti-microbial agent is between 10 mg and 800 mg. Preferably, the amount of the anti-microbial agent is between 25 mg and 300 mg. More preferably, the amount of the anti-microbial agent is 50 mg to 150 mg.

The present formulation relates to a solid suppository composition comprising: a) 4.8 wt % of clindamycin phosphate; b) 15.9 wt. % of a mixture of poloxamers; c) 63.5 wt. % of hydroxy fatty acid ester of polyethylene glycol; and d) 15.9 wt. % of water.

The suppository composition of this invention is characterized by the followings: (1) it exists as a solid form at room temperature (i.e., about 25° C.) and readily becomes a liquid-gel form at body temperature (e.g., about 37° C.) after vaginal or rectal administration; (2) it has the gel strength (i.e., exhibit a hysteresis loop behavior) to prevent leakage of the anti-microbial agent out from the vagina or anus and hence provide optimal absorption of the anti-microbial agent.

In accordance with the present invention, the present formulation includes a poloxamer mixture containing at least a first poloxamer polymer and a second poloxamer polymer. The first poloxamer polymer and the second poloxamer polymer are different and each has a CAS registration number of 9003-11-6 and the following chemical formula:
HO—(C₂H₄O)ₐ (C₃H₆O)_{b} (C₂H₄O)ₐ—H,
wherein 101≦a≦80 and 56≦b≦27, where “a” and “b” denote the number of poly-oxyethylene and poly-oxypropylene units, respectively.

The first poloxamer polymer may include HO—(C₂H₄O)ₐ(C₃H₆O)_{b}(C₂H₄O)ₐ—H where 101≦a≦80 and 56≦b≦27. Preferably, the first poloxamer polymer includes, but not limited to Poloxamer 188, Poloxamer 237, Poloxamer 338 and Poloxamer 407. More preferably, the first poloxamer polymer is poloxamer 407 (Lutrol® F127) or poloxamer 188 (Lutrol® F68). These poloxamer polymers are readily available from BASF (Louisiana, USA).

Similarly, the second poloxamer polymer may include HO—(C₂H₄O)ₐ(C₃H₆O)_{b}(C₂H₄O)ₐ—H where 101≦a≦80 and 56≦b≦27. Preferably, the second poloxamer polymer includes, but not limited to Poloxamer 188, Poloxamer 237, Poloxamer 338 and Poloxamer 407. More preferably, the second poloxamer polymer is poloxamer 407 (Lutrol® F127) or poloxamer 188 (Lutrol® F68).

The poloxamer mixture may further contain an additional poloxamer polymer. These additional poloxamers may also be selected from the group consisting of HO—(C₂H₄O)ₐ(C₃H₆O)_{b}(C₂H₄O)ₐ—H, where 101≦a≦80 and 56≦b≦27.

The poloxamer polymer includes, but not limited to, copolymers of ethylene oxide and propylene oxide. These polymers are represented by the following chemical structure and are available under various trade names, including Lutrol (BASF), Pluronic.RTM. series (BASF), Synperonic PE series (ICI); Emkalyx, Pluracare, and Plurodac.

The total poloxamer polymer (in the poloxamer mixture) is present in an amount of about 5% weight to about 30% weight. Preferably, the total poloxamer polymer is present in an amount of about 8% weight to about 17% weight. More preferably, the total poloxamer polymer is present in an amount of about 16% weight.

The present pharmaceutical formulation typically contains poloxamer mixture in the wt/wt ratio of about 1:1. Preferably, the poloxamer mixture is present in the amount of about 8% to about 17%. More preferably, the poloxamer mixture is present in the amount of about 16%.

In accordance with the present invention, the present formulation includes hydroxy fatty acid ester of polyethylene glycol. Preferably, fatty acid esters of polyethylene glycol wherein the polyethylene glycol has molecular weight between about 400 to about 1,000 daltons.

Preferably, hydroxy fatty acid ester of polyethylene glycol is polyethylene glycol 660 12 hydroxystearate (Solutol® HS 15, BASF Corporation).

Preferably, the hydroxy fatty acid ester of polyethylene glycol is polyethylene glycol 660 12 hydroxystearate and is present in the amount of about 40% to about 80%. More preferably, the hydroxy fatty acid ester of polyethylene glycol is present in the amount of about 60% to about 80%. More preferably, the hydroxy fatty acid ester of polyethylene glycol is present in the amount of about 65%.

The present invention provides a pharmaceutical formulation having a thermoreversible property for intra-vaginal and intra-rectal administration of an anti-microbial agent such as clindamycin which composition contains an anti-microbially effective amount of clindamycin phosphate dispersed in poloxamer and hydroxy fatty acid ester of polyethylene glycol.

The present pharmaceutical formulation may optionally contain water. If present, the formulation may contain water in the amount of about 5 wt % to about 30 wt %. Preferably, water is present in the amount of about 8 wt % to about 17 wt %.

The present pharmaceutical formulation may optionally contain stabilizer or absorption promoter. The stabilizer may include, but not limited to, benzyl alcohol paraben esters, ascorbyl palmitate, butylated hydroxyanisole, butylated hydroxytoluene and the like. Preferably, the stabilizer is butylated hydroxyanisole. If present, the formulation may contain a stabilizer in the amount of about 0.1 wt % to about 1 wt %. The absorption promoter may include, but not limited to, menthol, oleic acid, lecithin, taurocholate, glycocholate, limonene and the like. Preferably, the absorption promoter is oleic acid. If present, the formulation may contain an absorption enhancer in the amount of about 0.1 wt % to about 1 wt %.

Hysteresis Loop

Rheology is the science of the deformation and flow of matter when subject to an applied force. The magnitude of this applied force may range all the way from the gravitational force on a single, small, suspended particle to the very high shear rates encountered in high-speed mixing or homogenization. For water itself, for the common solvents, and for non-interacting liquid systems and solutions where the dissolved material is low in molecular weight, non-associating, and with limited solute-solvent interaction, or solvation, the characterization of flow is simple. When the shear stress is directly proportional to the shear rate applied, the system is said to be Newtonian. More complex solutions, however, tend to respond in a nonlinear manner to applied stress. When the dissolved or solvated molecules are large, the tendency to entangle and/or re-associate is high, and the solvent must exert some solvating force to maintain the polymer in solution. Such solutions are classified as non-Newtonian.

Viscosity is a measure of the force per unit area required to maintain a certain rate of flow. It is the internal friction of a flowing material and measures the tendency of the liquid to resist the applied shear force. Thus, viscosity is a property of all material capable of flowing. Shear stress (S) is defined as S=Force/Area, in dynes/cm², where Force is constant force and A is the area. Shear stress is measured in dynes/cm² and a dyne is the force needed to accelerate one gram of mass by one cm/sec/sec. Shear rate (D), also known as rate of deformation, is defined as velocity/x, in sec⁻¹, where x is the distance. The ratio of shear stress (S) to the shear rate (D) is the coefficient of viscosity, more commonly referred to simply as viscosity. The unit of viscosity measurement is the poise (1 dyne-sec/cm²) or centipoise (100 cP=l poise). Typical examples of viscosity are:

| Matter | Viscosity, poises |
|---|---|
| Water | 0.01 |
| Oils | 1-1,000 |
| Resins | 1,000-1,000,000 |

For the purposes of the present invention, viscosity is measured with rotational viscometers. A rotating body experiences a viscous drag, or retarding force, the amount of which varies with the speed of rotation. In rotational viscometers, the viscosity is determined by measuring the drag on a spindle rotating in the material. Main advantages of this instrument include: 1) they are simple to use; 2) continuous measurement can be made at a given rate of shear or stress; 3) the dependence of the viscosity on time can be readily determined; and 4) yield stresses can be determined. Specifically, Brookfield viscometer (Model DV-III plus Rheometer fitted with Rheocalc® application software) was used in the present studies.

One way of studying the flow behavior is to determine shear stress at various shear rates, as shown in FIG. 1. Here, the shear stress (S) is plotted versus the shear rate (D). In Newtonian flow, the shear stress and shear rate are always in direct proportion to each other. The viscosity of a Newtonian fluid will always be the same, regardless of the shear stress or shear rate. The upward and downward lines of the hysteresis loop for a Newtonian fluid will always be straight and overlap. There would be no hysteresis area. Under this condition, the solution of interest is said to be lacking a hysteresis loop behavior. Examples of Newtonian liquids are water, light oils or most Carbopol polymer gels and they exhibit little or no hysteresis loop behavior.

In non-Newtonian flow, the shear stress and shear rate are not in direct proportional to each other. The upward and downward lines of the hysteresis loop for non-Newtonian fluid will not be linear. As such, the lines do not overlap. There would always be a hysteresis area. Under this condition, the solution of interest is said to exhibit a hysteresis loop behavior (also known as thixotropy). Examples of non-Newtonian liquids include solution of hydroxyethylcellulose, hydroxypropylcellulose, carboxylmethyl cellulose and they exhibit a hysteresis loop behavior. Needless to say, thixotropy is a desirable characteristic in pharmaceutical gels. In this flow, the molecules at rest entangled together with the association of immobilized solvent. Under the influence of shear, the molecules tend to become disentangled and align themselves in the direction of flow. The molecules thus offer less resistance to flow.

Without wishing to be bound theory, it is believed that the hydroxy groups on the fatty acid esters of polyethylene glycol form hydrogen bonds with poloxamer polymer. The formation of the hydrogen bonds is believed to be crucial to render to the gelling and thermoreversiblility property to the present pharmaceutical formulation. It is further believed that the presence of poloxamer polymer (together with hydroxy fatty acid esters of polyethylene glycol) provides an optimal matrix vehicle to confer gelling and thermoreversible property to the pharmaceutical formulation.

In an aqueous solution, poloxamers undergo temperature induced sol-gel transitions. Typically, such solutions exists as liquids at temperatures below 20° C. and transform into gels at is higher temperatures depending on the type(s) of copolymers used, and such other factors as presence of cosolvents, salts, acids, drug particles, etc.

Without being bound by any theory, it is believed that the molecular mechanism(s) for these transitions relates to a multi-step process involving the entanglement of solvated polyoxyethylene chains, the dehydration of polyoxyethylene moieties and subsequent micellization to form aggregates with polyoxypropylene cores surrounded by polyoxyethylene coronas.

The thermoreversiblility of poloxamer transitions or aggregations has presented unique advantages for applications in various pharmaceutical dosage forms. However, for application as a suppository dosage form the need to exist as a solid at ambient temperature of about 25° C., complicates the use of such materials alone in the dosage form. Therefore the poloxamer-based system was augmented with additional material, in this case a hydroxy fatty acid esters of polyethylene glycol (e.g., Solutol® HS 15), to create a suppository formulation to overcome the deficiency of liquid suppositories.

Solutol® HS 15 is an off-white to faint yellow paste composed of a blend of polyethylene glycol 660 12-hydroxystearate and PEG 660 in the ratio 70:30 developed by BASF (Louisiana, USA). The material has a melting point of about 25° C. to about 30° C. The product has found application in neutraceutical formulations (BASF Technical Bulletin MEF 151 e April 1992, Register 5) and in experimental parenteral O/W emulsions formulations intended for targeted drug delivery systems. The material was selected for inclusion in this investigation because it is not a tri-glyceride material and has a low melting point of about 30° C. (not too far from physiological) and possesses terminal hydroxyl groups and internal oxyethylene groups to facilitate gel formation.

The anti-microbial pharmaceutical formulation presents as in a solid state at room temperature. The characteristics of the pharmaceutical composition include the features that the formulation is stable. Once inserted into a human body (i.e., exposed to a temperature of about 37° C.), the pharmaceutical formulation undergoes changes in the physical state and becomes a liquid-gel composition (i.e., the pharmaceutical formulation starts to melt and turns into a gel-like state). The solid state to gel-like state conversion provides optimal viscosity to the formulation, which permits better local delivery of the anti-microbial agents without leakage problems as in common commercial product (e.g., Hard fat suppository).

Without being further bound by any theory, it is believed that present pharmaceutical formulation has sufficient inter-molecular adhesive force to prevent leakage. The present pharmaceutical composition exhibits a hysteresis loop behavior, suggesting a non-Newtonian property and indicating that the shear stress and shear rate are not in direct proportional for the pharmaceutical composition. It is believed that the presence of hysteresis loop behavior, together with a proper viscosity value, offers the non-leakage advantage of the present pharmaceutical formulation.

Pharmaceutical Formulation

The present pharmaceutical formulation comprises a poloxamer mixture containing at least a first poloxamer polymer and a second poloxamer polymer, together with a hydroxy fatty acid ester of polyethylene glycol which has a thermoreversible feature suitable for suppository dosage forms. It is therefore a primary object of this invention to provide a pharmaceutical composition and a process for preparing the same and a method for treatment of microbial infections in vaginal or rectal areas. The thermoreversible pharmaceutical formulation provides a suppository formulation suitable to deliver an anti-microbial agent to these sites.

Preferably, anti-bacterial and anti-anti-fungal agents may be used in combination of the present pharmaceutical formulation. The present invention provides a drug delivery system for the treatment of bacterial vaginosis, vaginal candidiasis, genital herpes, chlamydiosis, trichomoniasis, gonorrhea and human papilloma virus or anorectal mucosa. The present formulation is demonstrated to be feasible and clinically effective.

The present invention provides an improved pharmaceutical formulation for use as a suppository dosage form. The formulation provides a method for using a hydrophilic matrix as a drug delivery system for both poorly soluble and high potent drugs. The present formulation is designed for administration and delivery of active pharmacological agents via the vaginal or rectal routes. Preferably, one suppository unit weighing from 2 to 3.5 g is administered via intra-vaginally or intra-rectally daily. Preferably, pharmaceutical formulation is administered 2.5 grams/day.

The present invention is illustrated by means of the following examples representative of the pharmaceutical formulations included in the present invention, which should not be considered as restrictions of the scope of the same.

### EXAMPLE 1

An anti-microbial pharmaceutical formulation having thermoreversible properties in accordance with the present invention was prepared. The pharmaceutical formulation has the following composition:

TABLE 1
Composition of a thermoreversible pharmaceutical formulation

| Components | % |
|---|---|
| Clindamycin phosphate | 2 |
| Poloxamer 407 | 8.2 |
| Poloxamer 188 | 8.2 |
| PEG 660 hydroxy fatty acid esters (Solutol ® HS 15) | 65.3 |
| Water | 16.3 |

### EXAMPLE 2

An additional anti-microbial pharmaceutical formulation having thermoreversible properties in accordance with the present invention was prepared. The pharmaceutical formulation has the following composition:

TABLE 2
Composition of a thermoreversible pharmaceutical formulation

| Components | % |
|---|---|
| Clindamycin phosphate | 2 |
| Poloxamer 407 | 4.1 |
| Poloxamer 188 | 12.3 |
| PEG 660 hydroxy fatty acid esters (Solutol ® HS 15) | 65.3 |
| Water | 16.3 |

### EXAMPLE 3

An additional anti-microbial pharmaceutical formulation with thermoreversible properties in accordance with the present invention was prepared and it has the following composition:

TABLE 3
Composition of a thermoreversible pharmaceutical formulation

| Components | % |
|---|---|
| Clindamycin phosphate | 2 |
| Poloxamer 407 | 12.3 |
| Poloxamer 188 | 4.1 |
| PEG 660 hydroxy fatty acid esters (Solutol ® HS 15) | 65.3 |
| Water | 16.3 |

### EXAMPLE 4

An additional anti-microbial pharmaceutical formulation with thermoreversible properties in accordance with the present invention was prepared and it has the following composition:

TABLE 4
Composition of a thermoreversible pharmaceutical formulation

| Components | % |
|---|---|
| Clindamycin phosphate | 2 |
| Poloxamer 407 | 6.1 |
| Poloxamer 188 | 6.1 |
| PEG 660 hydroxy fatty acid esters (Solutol ® HS 15) | 73.5 |
| Water | 12.2 |

### EXAMPLE 5

An additional anti-microbial pharmaceutical formulation with thermoreversible properties in accordance with the present invention was prepared and it has the following composition:

TABLE 5
Composition of a thermoreversible pharmaceutical formulation

| Components | % |
|---|---|
| Clindamycin phosphate | 4.8 |
| Poloxamer 407 | 7.9 |
| Poloxamer 188 | 7.9 |
| PEG 660 hydroxy fatty acid esters (Solutol ® HS 15) | 63.5 |
| Water | 15.9 |

### EXAMPLE 6

An additional anti-microbial pharmaceutical formulation with thermoreversible properties in accordance with the present invention was prepared and it has the following composition:

TABLE 6
Composition of a thermoreversible pharmaceutical formulation

| Components | % |
|---|---|
| Clindamycin phosphate | 4.8 |
| Poloxamer 407 | 4.0 |
| Poloxamer 188 | 11.9 |
| PEG 660 hydroxy fatty acid esters (Solutol ® HS 15) | 63.5 |
| Water | 15.8 |

### EXAMPLE 7

An additional anti-microbial pharmaceutical formulation with thermoreversible properties in accordance with the present invention was prepared and it has the following composition:

TABLE 7
Composition of a thermoreversible pharmaceutical formulation

| Components | % |
|---|---|
| Clindamycin phosphate | 4.8 |
| Poloxamer 407 | 11.9 |
| Poloxamer 188 | 4.0 |
| PEG 660 hydroxy fatty acid esters (Solutol ® HS 15) | 63.5 |
| Water | 15.8 |

### EXAMPLE 8

Physical Properties of Pharmaceutical Formulations

Physical State

Physical properties of the experimental batches containing various combinations of poloxamer and PEG 660 hydroxystearate were made. Physical state (e.g., solid or liquid-gel) of the pharmaceutical formulations of Examples 1-7 were examined after incubation at 37° C. for 30 minutes and were recorded in Table 8.

TABLE 8
Physical Properties of Thermoreversible
Pharmaceutical Formulations

Physical State

| Formulations | 25° C. | 37° C. |
|---|---|---|
| Example 1 | Solid | Liquid-gel |
| Example 2 | Solid | Liquid-gel |
| Example 3 | Solid | Liquid-gel |
| Example 4 | Solid | Liquid-gel |
| Example 5 | Solid | Liquid-gel |
| Example 6 | Solid | Liquid-gel |
| Example 7 | Solid | Liquid-gel |

As shown in Table 8, all seven pharmaceutical formulations exhibited thermoreversible properties. All seven pharmaceutical formulations were present in solid state at room temperature (i.e., about 25° C.). At this temperature, the pharmaceutical formulations were not pourable (i.e., flow upon within 10 minutes when slanting through 90 degrees). When exposed to the body temperature (i.e., about 37° C.), all the pharmaceutical formulations turned into liquid-gel. At this temperature, the pharmaceutical formulations were pourable (i.e., flow upon within 10 minutes when slanting through 90 degrees).

For the seven (7) pharmaceutical formulations, it is noted that both poloxamer 407 and poloxamer 188 range from about 4% to about 12% w/w; the ratio of poloxamer 407 to poloxamer 188 ranges from about 1:3 to about 3:1; and PEG 660 hydroxystearate is about 65% W/W.

### EXAMPLE 9

We prepared additional eighteen (18) pharmaceutical formulations (Examples 8-25) containing varying concentrations of poloxamers and PEG 660. Physical characterization (i.e., solid/liquid-gel) of these eighteen (18) additional pharmaceutical formulations were examined after incubation at 37° C. for 30 minutes and were recorded in Table 9.

TABLE 9
Physical Characterization of Additional 18 Thermoreversible
Pharmaceutical Formulations

| | | | PEG 660 | | | |
|---|---|---|---|---|---|---|
| | | | Hydroxy | | | |
| Formulation | Poloxamer | Poloxamer | stearate | Room | Body | Hysteresis |
| # | 407% w/w | 188% w/w | % w/w | Temperature | Temperature | Loop Area |
| Example 8 | 8.3 | 7.9 | 66.7 | Solid | Liquid-gel | Present |
| Example 9 | 8.3 | 11.9 | 50.0 | Solid | Liquid-gel | Present |
| Example 10 | 11.1 | 11.1 | 55.6 | Solid | Liquid-gel | Present |
| Example 11 | 11.1 | 19.5 | 38.9 | Solid | Liquid-gel | Present |
| Example 12 | 13.9 | 13.9 | 44.4 | Solid | Liquid-gel | Present |
| Example 13 | 16.7 | 8.3 | 50.0 | Solid | Liquid-gel | Present |
| Example 14 | 0 | 0 | 100 | Solid | Liquid | Absent |
| Example 15 | 0 | 50.0 | 0 | Solid | Solid | Absent |
| Example 16 | 50.0 | 0 | 0 | Solid | Solid | N/A |
| Example 17 | 0 | 25.0 | 50.0 | Solid | Liquid | Absent |
| Example 18 | 25.0 | 0 | 50.0 | Solid | Solid | N/A |
| Example 19 | 25.0 | 25.0 | 0 | Solid | Solid | N/A |
| Example 20 | 8.3 | 25.0 | 33.3 | Solid | Solid | N/A |
| Example 21 | 8.3 | 33.3 | 16.7 | Solid | Solid | N/A |
| Example 22 | 16.7 | 16.7 | 33.3 | Solid | Solid | N/A |
| Example 23 | 19.5 | 11.1 | 38.9 | Solid | Solid | N/A |
| Example 24 | 25.0 | 8.3 | 33.3 | Solid | Solid | N/A |
| Example 25 | 33.3 | 8.3 | 16.7 | Solid | Solid | N/A |

N/A is not applicable (i.e., hysteresis loop area cannot be determined in a solid)

Table 9 shows that six (6) of the additional pharmaceutical formulations (i.e., Examples 8-13) exhibited thermoreversible property as well as hysteresis loop behavior.

With respect to pharmaceutical formulations of Examples 8-13, it is noted that both poloxamer 407 and poloxamer 188 range from about 8% to about 20% w/w; the ratio of poloxamer 407 to poloxamer 188 ranges from about 1:0.5 to about 1:2; and PEG 660 hydroxy stearate ranges from about 40% to about 70% w/w.

Table 9 also shows that the twelve (12) additional pharmaceutical formulations (i.e., Examples 15-25) do not exhibit thermoreversible property (i.e., do not exhibit a hysteresis loop behavior), indicating specificity.

The absence of either poloxamer 407 (Examples 15 and 17) or poloxamer 188 (Examples 16 and 19) were found unsuitable because they formed solid or did not exhibit a hysteresis loop behavior at 37° C. Also, example 17 did not form a solid at ambient temperature. These formulations were found not to have proper viscosity values and did not exhibit hysteresis loop behavior. In addition, when the amount of PEG 660 hydroxy stearate was not optimal (Examples 21 and 25), the pharmaceutical formulations also did not exhibit a hysteresis loop behavior and thermoreversible property. Accordingly, although being solid at 25° C., they failed to transform into pourable liquid-gel when incubated at 37° C. within 30 minutes.

Rheological Studies

Rheological testing (i.e., measurement of viscosity) of the prepared pharmaceutical formulations (i.e., Examples 8-25) described previously was performed using a Brookfield DV-III+ Rheometer and appropriate T-bar spindles with the Helipath stand. The viscosity measurements were done on the pharmaceutical formulations after incubation at 25° C. and 37° C., respectively for 30 minutes. The operating range of the measure instrument is >440,000 cP. The data are summarized in Table 10.

TABLE 10
Viscosity of Thermoreversible Pharmaceutical Formulations

| | | | PEG 660 | | | |
|---|---|---|---|---|---|---|
| | | | Hydroxy | Viscosity, | Viscosity, | |
| Formulation | Poloxamer | Poloxamer | Stearate | cP Room | cP Body | Hysteresis |
| # | 407% w/w | 188% w/w | % w/w | Temperature | Temperature | Loop Area |
| Example 8 | 8.3 | 7.9 | 66.7 | 22,252 | 1,984 | Present |
| Example 9 | 8.3 | 11.9 | 50.0 | 4,888 | 2,392 | Present |
| Example 10 | 11.1 | 11.1 | 55.6 | 5,088 | 1,928 | Present |
| Example 11 | 11.1 | 19.5 | 38.9 | 16,304 | 6,304 | Present |
| Example 12 | 13.9 | 13.9 | 44.4 | 12,289 | 5,340 | Present |
| Example 13 | 16.7 | 8.3 | 50.0 | 8,936 | 5,340 | Present |
| Example 14 | 0 | 0 | 100 | 440,000 | 1,280 | Absent |
| Example 15 | 0 | 50.0 | 0 | 444,000 | 400,000 | N/A |
| Example 16 | 50.0 | 0 | 0 | 386,900 | 430,440 | N/A |
| Example 17 | 0 | 25.0 | 50.0 | 3,804 | 1,960 | Absent |
| Example 18 | 25.0 | 0 | 50.0 | 100,560 | 113,200 | N/A |
| Example 19 | 25.0 | 25.0 | 0 | 417,960 | 393,920 | N/A |
| Example 20 | 8.3 | 25.0 | 33.3 | 57,400 | 38,760 | N/A |
| Example 21 | 8.3 | 33.3 | 16.7 | 385,560 | 347,920 | N/A |
| Example 22 | 16.7 | 16.7 | 33.3 | 212,160 | 190,270 | N/A |
| Example 23 | 19.5 | 11.1 | 38.9 | 108,880 | 46,660 | N/A |
| Example 24 | 25.0 | 8.3 | 33.3 | 124,520 | 138,600 | N/A |
| Example 25 | 33.3 | 8.3 | 16.7 | 400,920 | 347,520 | N/A |

N/A is not applicable (i.e., hysteresis loop area cannot be determined in a solid)

The data presented in Table 10 reveal an interesting result. One of the desirable goal is to obtain a pharmaceutical composition that is a solid (not pourable when slanted through 90 degrees) at room temperature and a liquid-gel (pourable when slanted through 90 degrees) at body temperature. Accordingly, it is desirable that the pharmaceutical composition shall have a proper high viscosity value at 25° C. and have a proper low viscosity value at 37° C. At the same time, it is essential that the pharmaceutical composition shall exhibit a hysteresis loop behavior.

In the absence of either poloxamer 407 (Examples 14, 15, and 17) or poloxamer 188 (Examples 14, 16, and 19), the formulations were found not suitable because they either formed a solid with too high viscosity values at 37° C. or a liquid at room temperature. When the amount of PEG 660 hydroxy stearate was not optimal (Examples 21 and 25), the pharmaceutical formulations did not exhibit thermoreversible property (i.e., transformed into liquid-gel that is pourable when slanted through 90 degrees) when incubated at 37° C. within 30 minutes and did not exhibit a hysteresis loop behavior. In addition, they failed to have a proper viscosity value.

With respect to the pharmaceutical compositions of Examples 8-13, these pharmaceutical formulations presented as a solid at 25° C. and transformed into a liquid-gel when incubated at 37° C. within 30 minutes. These pharmaceutical compositions are characterized by: i) total poloxamer is less than about 30%, ii) poloxamer 407 and poloxamer 188 range from about 8% to about 20%; iii) the ratio of poloxamer 407 to poloxamer 188 ranges from about 1:0.5 to about 1:2; and iv) PEG 660 hydroxy stearate ranges from about 40% to about 70%,

FIG. 1 shows various pharmaceutical formulations that exhibit hysteresis loop behavior. A hysteresis loop area is a useful way of visualizing gel behavior due to associative molecular interactions of the vehicle components. The presence of hysteresis loop area demonstrates that the liquid-gel formed upon melting at body temperature is expected to resist leakage.

In contrast, control pharmaceutical formulations do not exhibit hysteresis loop behavior. Hard fat suppository is a commercial product containing clindamycin and it does not exhibit a hysteresis loop behavior (See, #6 in FIG. 1). As such, the Hard fat suppository results in leakage from vaginal vault and poses a major disadvantage. Pharmaceutical formulations containing only PEG 600 hydroxy stearate (without any poloxamer polymer) or a poloxamer mixture of two poloxamer polymers (without PEG hydroxy stearate) also did not exhibit a hysteresis loop behavior (See, # 4 and #5, respectively in FIG. 1).

The effects of drug incorporation on the pharmaceutical formulation viscosity were further examined. Anti-microbial agent (2-10 wt %) may be added to the pharmaceutical formulation without adversely affecting the viscosity of the formulation. Addition of drug (i.e., anti-microbial agent) generally leads to a slight increase in viscosity of the pharmaceutical formulations.

Additional rheological examinations were performed for pharmaceutical formulations of Examples 5, 6 and 7. The data are shown in Table 11. These pharmaceutical formulations contain 4.8% clindamycin phosphate.

TABLE 11
Composition and Viscosity of Pharmaceutical Formulation
of Examples 5, 6, and 7 Containing Clindamycin Phosphate

| | | | PEG 660 | | |
|---|---|---|---|---|---|
| | | | Hydroxy | Clindamycin | Viscosity, |
| Formulation | Poloxamer | Poloxamer | stearate | Phosphate | cP Body |
| # | 407% w/w | 188% w/w | % w/w | % w/w | Temperature |
| Example 5 | 7.9 | 7.9 | 63.5 | 4.8 | 3,404 |
| Example 6 | 4.0 | 11.9 | 63.5 | 4.8 | 1,972 |
| Example 7 | 11.9 | 4.0 | 63.5 | 4.8 | 4,520 |

The pharmaceutical formulations of Examples 5, 6 and 7 all exhibited thermoreversible property and hysteresis loop behavior. Specifically, these formulations formed a solid at 25° C. and transformed into pourable liquid-gel (with viscosity <5,000 cP) when incubated at 37° C. within 30 minutes.

The disclosures of the cited publications are incorporated herein in their entireties by reference. It is to be understood, however, that the scope of the present invention is not to be limited to the specific embodiments described above. The invention may be practiced other than as particularly described and still be within the scope of the accompanying claims.

What is claimed is:

1. A thermoreversible pharmaceutical formulation, comprising:
a) an anti-microbial agent;
b) a poloxamer mixture containing a first poloxamer polymer and a second poloxamer polymer, wherein the first poloxamer polymer and the second poloxamer polymer are not the same, and wherein the first and the second poloxamer polymers are polymers represented by the chemical structure of:
HO—(C₂H₄O)ₐ(C₃H₆O)_{b}(C₂H₄O)ₐ—H,
where 101≦a≦80 and 56≦b≦27, and “a” and “b” denote the number of poly-oxyethylene and poly-oxypropylene units, respectively; and
c) a hydroxy fatty acid ester of polyethylene glycol,
wherein the pharmaceutical formulation having a viscosity of about 8,500 cP to about 400,000 cP at room temperature, a viscosity of about 1,000 cP to about 8,000 cP at body temperature, and exhibiting a hysteresis loop behavior.

2. The thermoreversible pharmaceutical formulation of claim 1, wherein the formulation has a viscosity of about 8,500 cP to about 25,000 cP at room temperature, and a viscosity of about 1,000 cP to about 5,000 cP at body temperature.

3. The thermoreversible pharmaceutical formulation of claim 1, wherein the formulation of has a viscosity of about 8,500 cP to about 25,000 cP at room temperature and has a viscosity of about 1,500 cP to about 3,000 cP at body temperature.

4. The thermoreversible pharmaceutical formulation of claim 1, wherein the anti-microbial agent is selected from the group consisting of an anti-bacterial agent, an anti-fungal agent and an anti-yeast agent.

5. The thermoreversible pharmaceutical formulation of claim 4, wherein the anti-bacterial agent is at least one compound selected from the group consisting of clindamycin, fluconazole, flucytosine, itraconazole, ketoconazole, miconazole, ciclopirox, clotrimazole, econazole, nystatin, oxiconazole, terbinafine, tioconazole, butoconazle, terconazole, metronidazole, and isoconazole.

6. The thermoreversible pharmaceutical formulation of claim 4, wherein the anti-bacterial agent is clindamycin.

7. The thermoreversible pharmaceutical formulation of claim 4, wherein the anti-bacterial agent is clindamycin phosphate.

8. The thermoreversible pharmaceutical formulation of claim 1, wherein the anti-microbial agent is present in the amount of about 0.1 wt % to about 10 wt %.

9. The thermoreversible pharmaceutical formulation of claim 1, wherein the anti-microbial agent is present in the amount of about 2 wt % to about 5 wt %.

10. The thermoreversible pharmaceutical formulation of claim 1, wherein the anti-microbial agent is present in the amount of about 4.8 wt %.

11. The thermoreversible pharmaceutical formulation of claim 1, wherein the anti-microbial agent is an anti-fungal agent.

12. The thermoreversible pharmaceutical formulation of claim 11, wherein the anti-fungal agent is selected from the group consisting of clotrimzole, fluconazole, flucytosine, itraconazole, ketoconazole, miconazole, ciclopirox, econazole, nystatin, oxiconazole, terbinafine, tioconazole, butoconazle, terconazole, metronidazole, isoconazole, and tolnaftate.

13. The thermoreversible pharmaceutical formulation of claim 11, wherein the anti-fungal agent is clotrimazole.

14. The thermoreversible pharmaceutical formulation of claim 1, further comprising a stabilizer or an absorption promoter.

15. The thermoreversible pharmaceutical formulation of claim 14, wherein the stabilizer is selected from the group of benzyl alcohol paraben esters, ascorbyl palmitate, butylated hydroxyanisole, and butylated hydroxytoluene.

16. The thermoreversible pharmaceutical formulation of claim 14, wherein the stabilizer is butylated hydroxyanisole.

17. The thermoreversible pharmaceutical formulation of claim 14, wherein the absorption promoter is selected from the group consisting of menthol, oleic acid, lecithin, taurocholate, glycocholate, and limonene.

18. The thermoreversible pharmaceutical formulation of claim 14, wherein the absorption promoter is oleic acid.

19. The thermoreversible pharmaceutical formulation of claim 14, wherein the stabilizer in the amount of about 0.1 wt % to about 1 wt %.

20. The thermoreversible pharmaceutical formulation of claim 14, wherein the absorption promoter is in the amount of about 0.1 wt % to about 1 wt %.

21. The thermoreversible pharmaceutical formulation of claim 1, wherein the poloxamer is poloxamer 188, poloxamer 237, poloxamer 338, or poloxamer 407.

22. The thermoreversible pharmaceutical formulation of claim 1, wherein the poloxamer is poloxamer 407 or poloxamer 188.

23. The thermoreversible pharmaceutical formulation of claim 1, wherein the poloxamer polymer is present in the amount of about 5 wt % to 30 wt %.

24. The thermoreversible pharmaceutical formulation of claim 1, wherein the poloxamer polymer is present in the amount of about 8 wt % to about 17 wt %.

25. The thermoreversible pharmaceutical formulation of claim 1, wherein the poloxamer polymer is present in the amount of about 16 wt %.

26. The thermoreversible pharmaceutical formulation of claim 1, wherein the first poloxamer polymer and the second poloxamer polymer is present in a wt/wt ratio of about 1:0.125 to about 1:1.

27. The thermoreversible pharmaceutical formulation of claim 1, wherein the first poloxamer polymer and the second poloxamer polymer is present in a wt/wt ratio of about 1:1.

28. The thermoreversible pharmaceutical formulation of claim 1, wherein the hydroxy fatty acid ester of polyethylene glycol is polyethylene glycol 660 hydroxystearate.

29. The thermoreversible pharmaceutical formulation of claim 1, wherein the hydroxy fatty acid ester of polyethylene glycol has molecular weight between about 400 to about 1,000 daltons.

30. The thermoreversible pharmaceutical formulation of claim 1, wherein the hydroxy fatty acid ester of polyethylene glycol is present in the amount of about 40 wt % to about 80 wt %.

31. The thermoreversible pharmaceutical formulation of claim 1, wherein the hydroxy fatty acid ester of polyethylene glycol is present in the amount of about 60 wt % to about 80 wt %.

32. The thermoreversible pharmaceutical formulation of claim 1, wherein the hydroxy fatty acid ester of polyethylene glycol is present in the amount of about 65 wt %.

33. The thermoreversible pharmaceutical formulation of claim 1, further comprising water.

34. The thermoreversible pharmaceutical formulation of claim 33, wherein water is present in the amount of about 5 wt % to about 30 wt %.

35. The thermoreversible pharmaceutical formulation of claim 33, wherein water is present in the amount of about 8 wt % to about 17 wt %.

36. A pharmaceutical formulation having a thermoreversible property, comprising:
a) clindamycin phosphate;
b) a poloxamer mixture containing a first poloxamer polymer and a second poloxamer polymer, wherein the first poloxamer polymer and the second polaxamer polymer are not the same, and wherein the first poloxamer polymer is poloxamer 407 and the second poloxamer polymer is poloxamer 188; and
c) polyethylene glycol 660 hydroxystearate,
wherein the pharmaceutical formulation having a viscosity of about 8,500 cP to about 400,000 cP at room temperature, a viscosity of about 1,000 cP to about 8,000 cP at body temperature, and exhibiting a hysteresis loop behavior.

37. A process of preparing a pharmaceutical formulation having a thermoreversible property, comprising the steps of:
a) preparing a poloxamer mixture containing at least a first poloxamer and a second poloxamer polymer;
b) preparing a molten solution of hydroxy fatty acid ester of polyethylene glycol;
c) adding the poloxamer mixture to the molten solution of hydroxy fatty acid ester of polyethylene glycol; and
d) adding an anti-microbial agent to form a thermoreversible pharmaceutical formulation.

38. The process of claim 37, wherein said first poloxamer is selected from the group containing a block copolymer of polyoxyethylene and polyoxypropylene, wherein the poloxamer is represented by the chemical structure of:
HO—(C₂H₄O)ₐ(C₃H₆O)_{b}(C₂H₄O)ₐ—H,
where 101≦a≦80 and 56≦b≦27, and “a” and “b” denote the number of poly-oxyethylene and poly-oxypropylene units, respectively.

39. The process of claim 37, wherein said second poloxamer is selected from the group containing a block copolymer of polyoxyethylene and polyoxypropylene, wherein the poloxamer is represented by the chemical structure of:
HO—(C₂H₄O)ₐ(C₃H₆O)_{b}(c₂H₄O)ₐ—H,
where 101≦a≦80 and 56≦b≦27, and “a” and “b” denote the number of poly-oxyethylene and poly-oxypropylene units, respectively.

40. The process of claim 37, wherein the poloxamer mixture is prepared by heating at a temperature of 60° C. to about 75° C.

41. The process of claim 37, wherein the poloxamer mixture is prepared by heating at a temperature of about 65° C.

42. The process of claim 37, wherein the preparing step of hydroxy fatty acid ester of polyethylene glycol is carried out at about 60° C. to about 70° C.

43. The process of claim 37, wherein the anti-microbial agent is an anti-bacterial agent.

44. The process of claim 37, wherein the anti-bacterial agent is clindamycin.

45. The process of claim 37, wherein the anti-bacterial agent is clindamycin phosphate.

46. A method for treating a microbial infection in a mammal, comprising the step of administering to a mammal a therapeutically effective amount of the thermoreversible pharmaceutical formulation of claim 1.

47. The method of claim 46, wherein anti-microbial is clindamycin.

48. The method of claim 46, wherein the anti-microbial is clindamycin phosphate.

49. The method of claim 46, wherein the mammal is a human.

50. The method of claim 46, wherein the human is a female.

51. The method of claim 46, wherein the thermoreversible pharmaceutical formulation is administered via intra-vaginally or intra-rectally.

52. The method of claim 46, wherein the thermoreversible pharmaceutical formulation is administered from about 2 to about 3.5 grams/day.

53. The method of claim 46, wherein the thermoreversible pharmaceutical formulation is administered about 2.5 grams/day.
